(19) United States
(12) Patent Application Publication
(10) Pub. No.: US 2026/0295171 A1
(43) Pub. Date: Oct. 1, 2026

(54) DRUG DELIVERY DEVICE

(71) Applicant: SANOFI, Paris (FR)

(72) Inventors: Uwe DASBACH, Frankfurt am Main (DE); Claus GEIGER, Frankfurt am Main (DE); Tim GLÄSSER, Rüsselsheim (DE); Moritz KEIM, Frankfurt am Main (DE); Bernd KÜHN, Frankfurt (DE); Matthias RAU, Rüsselsheim (DE); Marc SCHADER, Frankfurt am Main (DE)

(21) Appl. No.: 19/490,266

(22) PCT Filed: Jun. 5, 2024
(86) PCT No.: PCT/EP2024/065437

§ 371 Date: Dec. 4, 2025

(30) Foreign Application Priority Data
Jun. 5, 2023 (EP) 23315228.9

(51) Int. Cl. A61M 5/315; A61M 5/20; A61M 5/32

(52) U.S. Cl. CPC A61M 5/31511; A61M 5/20; A61M 5/31535; A61M 5/31571; A61M 5/31581; A61M 5/3257; A61M 2005/2013; A61M 2005/2026; A61M 2005/206; A61M 2005/208; A61M 2005/31518; A61M 2005/3152; A61M 2205/273; A61M 2205/8281

(57) ABSTRACT

A drug delivery device comprising a housing; a drug container within the housing having a chamber with a dispensing end and a remote end; a stopper within the chamber that sealingly closes the chamber at the remote end to enclose a drug; and a plunger arrangement including multiple plunger elements configured to move the stopper in a dispensing direction toward the dispensing end. The plunger elements are rotatable about an axis perpendicular to the dispensing direction and are successively introducible into the chamber to contact and push the stopper in the dispensing direction upon rotation about the axis.

### CROSS-REFERENCE TO RELATED APPLICATIONS

The present application is the national stage entry of International Patent Application No. PCT/EP2024/065437, filed on Jun. 5, 2024, and claims priority to European Application No. EP 23315228.9, filed on Jun. 5, 2023, the disclosures of which are incorporated herein by reference.

### TECHNICAL FIELD

The present disclosure relates to a drug delivery device, in particular a very compact and flat drug delivery device.

### BACKGROUND

In drug delivery devices, drug is often delivered to a user via a needle which pierces the skin of the user (or patient). The drug may be accommodated within a drug container of the drug delivery device, e.g. within a syringe arranged within the drug delivery device. Conventional drug delivery devices comprising syringes and an associated drive mechanism have a shape basically corresponding to the shape of the syringe. In particular, conventional drug delivery devices comprising syringes have an elongated cylindrical shape, wherein an axis of the drug delivery device may correspond to an axis of the needle. Such a drug delivery device may often be referred to as pen-type device. A needle of the syringe may be protected by a needle sleeve, a needle shield, and/or a cap of the drug delivery device.

Such pen-type devices usually have a small bearing surface for being in contact with the user, e.g. on the skin in the vicinity of an injection site. In particular, in most cases, an axial end face of the pen-type device may be considered for being in contact with the user during injection of the corresponding dose. However, such an end face provides a very small basis for supporting the pen-type device. Further, the cylindrical form may be hard to handle, in particular if the user has some motoric impairment.

### SUMMARY

It is an object of the present disclosure to facilitate improvements associated with drug delivery devices, particularly with respect to size, shape and operability.

This object is achieved by the disclosed subject-matter, for example by the subject-matter defined in the appended independent claim. Advantageous refinements and developments are subject to dependent claims and/or set forth in the description below.

One aspect of the present disclosure relates to a drug delivery device, comprising: a housing; a drug container within the housing, the drug container having a chamber, a dispensing end, and a remote end facing away from the dispensing end; a stopper within the chamber, wherein the stopper sealingly closes the chamber of the drug container on the side of the remote end; a drug within the chamber, wherein the drug is enclosed by the drug container and the stopper; and a plunger arrangement having several plunger elements for moving the stopper in a dispensing direction towards the dispensing end of the drug container, wherein the plunger elements are configured for being rotated around an axis perpendicular to the dispensing direction, for being introduced into the chamber of the drug container one after the other and for pushing the stopper in the dispensing direction, upon rotation of the plunger elements around the axis.

The plunger arrangement comprising several plunger elements being rotatable around the axis and being introducible into the drug container enables a very compact design and easy operability of the drug delivery device. In particular, because of the plunger elements being movable relative to each other and around the axis, less space is needed in the dispensing direction. For example, the housing of the very compact drug delivery device may be (computer-)mouse-shaped. Such a mouse-shaped drug delivery device may be handled, e.g. gripped, easily and comfortably. In addition, a bearing surface of such a mouse-shaped drug delivery device may correspond to a bottom surface of the correspondingly mouse-shaped housing and may be much larger than a bearing surface of a conventional pen-type drug delivery device, with the bearing surface being configured to be in direct contact with a skin of the user during injection of the drug.

The drug delivery device may be a fully functional drug delivery device. The drug may be a medicament. The drug delivery device may be an autoinjector. In an autoinjector the energy for the drug delivery operation may be prestored in an energy storage member. That is to say, the user does not have to provide the energy for the drug delivery operation, e.g. when preparing the drug delivery device for use. Rather, this energy may be preloaded into the system by the manufacturer. For example, a drive spring, e.g. a spiral spring or flat spiral spring, may be pre-stressed or pre-biased to provide the energy for the drug delivery operation.

The housing may have the shape of a (computer-)mouse or similar to a (computer-)mouse. One advantage of this mouse-shape is the flat and compact design. The flat design enlarges the contact area on the skin, i.e. a bearing surface, for the injection, which makes the device more stable in place. The bearing surface may be a part of the housing and may be configured to be in contact with the injection site, in particular the skin of the user, during usage of the drug delivery device. In case of the mouse-shaped housing, the bearing surface may be a bottom surface of the housing. The bearing surface may be perpendicular to the axis. The bearing surface may be parallel to the dispensing direction. A height of the housing parallel to the axis may be smaller than a length of the housing perpendicular to the axis. The bearing surface of the drug delivery device may be adjacent to a location where the needle pierces the skin for the drug delivery operation when the drug delivery device is used for the drug delivery operation. There may be a grip area opposite to the bearing surface, i.e. facing away from the skin of the user during usage. The mouse-design enables to provide a very large grip area compared to a cylindrical drug delivery device. The large grip area also makes the drug delivery device easier to hold for patients with limited dexterity, as is the case with rheumatic patients, for example. The compact design is made possible by the plunger elements rotatable around the axis.

In one embodiment, the plunger elements are configured such that a first plunger element of the plunger elements pushes the stopper in the dispensing direction and that a second plunger element of the plunger elements pushes the first plunger element towards the stopper, upon rotation of the plunger elements around the axis. In other words, the second plunger element on the first plunger element acts and the first plunger element acts on the stopper. In general, if there are more than two plunger elements, each plunger elements acts on the preceding plunger element, wherein the first plunger element acts on the stopper. The plunger elements movable relative to each other and acting on each other enable a very compact design of the drug delivery device.

In one embodiment, the plunger elements are coupled to each other at sides of the plunger elements facing the axis. The plunger elements may be hingedly coupled to each other. For example, two neighboring plunger elements may be coupled to each other by a corresponding hinge. The hinge may be formed from the same material as the plunger elements. For example, the plunger elements and the hinges may be made from one piece. The hinges may be film hinges or living hinges. The coupled plunger elements may form a flexible plunger.

In one embodiment, the drug delivery device, comprises a needle for injecting the drug into an injection site, wherein the needle is communicatively coupled to the drug container at the dispensing end of the drug container. The needle may be in or may be brought into fluid communication with an interior of the drug container. The needle may be integrated into the drug container. The medicament, e.g. a liquid medicament, is expediently arranged in the interior of the drug container. The drug container may be a syringe, e.g. a syringe with a preinstalled needle, such as a staked needle. In other embodiments, the medicament container may be a cartridge, which may have to be brought into fluid communication with the needle, e.g. by piercing a cartridge septum with the needle. The drug delivery device may be configured such that the needle is not movable relative to the housing and/or the drug container. The needle may be protected by a needle sleeve. The needle sleeve may be movable parallel to the axis. The drug delivery device may be configured such that when the needle sleeve is displaced from an initial or first position to a trigger or second position, the drug delivery device automatically initializes the drug delivery operation. A surface of the needle sleeve being configured to be in direct contact with the skin of the user during usage of the drug delivery device may be much smaller than the bearing surface of the drug delivery device. Therefore, the bearing surface may provide a very stable base for using the drug delivery device.

In one embodiment, the needle extends in a direction parallel to the axis. So, the needle may extend perpendicular to the dispensing direction. The needle may be configured for piercing the skin of the user. Optionally, the drug delivery device may comprise a second needle. In this context, the needle piercing the skin of the user may be referred to as first needle and the further needle may be referred to as second needle. The first needle may communicate with the second needly, e.g. by a conduit, for guiding the drug from the drug container through the second needle towards the first needle. The second needle may extend perpendicular to the first needle and/or the axis. The second needle may extend in parallel to the dispensing direction. The conduit may comprise a bended portion, e.g. a 90° bended portion.

In one embodiment, the drug delivery device comprises a rotatable member, which is rotatable around the axis and which is coupled to the plunger elements such that the rotatable member rotates the plunger elements around the axis upon rotation of the rotatable member. The rotatable member enables to rotate the plunger elements around the axis and as such to push the stopper in the dispensing direction in a very easy way.

In one embodiment, the rotatable member comprises a gear wheel and the plunger elements each comprise a toothed portion, with the tooth portion of the plunger elements being configured to engage with the gear wheel, upon rotation of the rotatable member around the axis. The gear wheel, which may be rigidly fixed to the rotatable member and which is configured to be rotated together with the rotatable member, easily enables to couple the rotatable member with the plunger elements.

In one embodiment, the rotatable member comprises several protrusions at a surface of the rotatable member, with the surface facing the plunger elements and being perpendicular to the axis, and the plunger elements each comprise an indentation, with the protrusions of the rotatable member being configured to engage with the indentation of the corresponding plunger element, upon rotation of the rotatable member around the axis. The function of the protrusions and the indentations corresponds to the function of the gear wheel and, respectively, the toothed portions. The protrusions and the indentations may be arranged additionally or alternatively to the gear wheel and the toothed portions.

In one embodiment, the drug delivery device comprises the energy storage member coupled to the rotatable member and configured for rotating the rotatable member, wherein the energy storage member is loaded in an initial state. For example, the energy storage member may be a spiral spring, e.g. a flat spiral spring. The flat spiral spring may be arranged parallel to the rotatable member. Alternatively or additionally, an axis of the spiral spring may correspond to the above axis. The flat spiral spring may be attached to and/or may be arranged within a recess of the rotatable member. For example, the rotatable member comprises a hollow base accompanying the energy storage member. In case of the spiral spring or flat spiral spring the energy storage member may be pre-loaded by biasing the spiral spring or, respectively, the flat spiral spring. The energy storage member may allow for a given activation dynamic.

In one embodiment, the drug delivery device comprises a release mechanism being configured for locking the rotatable member in the initial state and for releasing the rotatable member such that the energy storage member rotates the rotatable member around the axis, upon activation of the release mechanism. The release mechanism may comprise an activation member which may have to be moved relative to the housing in order to enable triggering of the drug delivery operation or to trigger the drug delivery operation. The activation member may be a rod. In other embodiments, the needle sleeve may be configured as the activation member. If the needle sleeve is the activation member, the drug delivery operation may be triggered without interacting with, e.g. without pushing, any further activation member, e.g. the rod, in addition to the needle sleeve. In other words, if the needle sleeve is the activation member, the drug delivery operation may be triggered by the needle sleeve, e.g. by movement thereof. Enable triggering of the drug delivery operation may comprise that in addition to movement of the activation member another member such as a trigger member has to be actuated, e.g. a button, has to be pressed. In other embodiments, the activation member itself may comprise the trigger member, wherein one axial end of the rod may be used as the button. The rod may comprise one or more recesses acting in conjunction with corresponding pins or ribs for arresting or releasing the energy storage member. The rod may extend parallel to the dispensing direction and/or perpendicular to the axis. The direction, in which the rod extends and/or may be moved, is not perfectly parallel to the dispensing direction. Therefore, in this description, the direction, in which the rod extends and/or may be moved, is referred to as “basically” the dispensing direction. In other embodiments, the rod may extend perpendicular to the dispensing direction and/or parallel to the axis. When the release mechanism is activated, the drug delivery operation may be initiated, e.g. resulting in the stopper being moved in the dispensing direction relative to the housing.

In one embodiment, the release mechanism comprises the rod, which is configured for blocking an arrestor rib of the rotatable member in the initial state and for releasing the arrestor rib upon moving the rod. The rod may be moved basically in the dispensing direction for releasing the arrestor rib. The activation of the release mechanism may involve a user contact force exerted on the rod, e.g. on the button coupled to rod. The release mechanism may be activated by pushing the rod and/or the button basically in the dispensing direction. For example, the rod comprises a recess for accommodating and as such releasing the arrestor upon moving the rod in the dispensing direction.

In one embodiment, the drug delivery device comprises a needle sleeve for protecting the needle, wherein the needle sleeve is operatively coupled to the release mechanism and configured such that an activation of the release mechanism is prevented as long as the needle sleeve protects the needle and that the activation of the release mechanism is enabled, when the needle sleeve exposes the needle. The needle sleeve may be movable relative to the housing. The needle sleeve may block the activation of the release mechanism in a first position of the needle sleeve relative to the housing and may release the activation of the release mechanism in a second position of the needle sleeve relative to the housing.

The needle sleeve may be provided to cover the needle, in particular the first needle for piercing the skin. The needle sleeve may be provided to cover the needle before the needle pierces the skin and/or after the needle has been removed from the skin, e.g. after completion of the drug delivery operation. Before the drug delivery operation is commenced, the needle sleeve may protrude from the housing, e.g. to cover the tip of the needle (such as by axially extending beyond the tip of needle, e.g. at least with a contact surface of the needle sleeve). For the drug delivery operation, the needle sleeve may be displaced relative to the housing. After completion of the drug delivery operation, the needle sleeve may be moved relative to the housing, e.g. to cover the tip of needle and/or into a third position relative to the housing. The drug delivery device may comprise a sleeve spring. The sleeve spring may be operatively couplable to or coupled to the needle sleeve in order to move the needle sleeve, e.g. into the axial direction relative to the housing. The force of the needle sleeve spring may have to be overcome in order to move the needle sleeve into the housing. In a final position, e.g. after the drug delivery operation has been completed and the drug delivery device has been removed from the skin, the needle sleeve may be locked against a further movement with respect to the housing, such as by a locking mechanism. This may contribute to a safe handling of the drug delivery device after its usage by protecting the used needle.

In one embodiment, the drug delivery device comprises one or more engaging features configured to engage with the release mechanism and to lock the release mechanism in a final state after the drug is injected. For example, the engaging features engage with the rod of the release mechanism in the final state. This may contribute to a safe handling of the drug delivery device after its usage.

In one embodiment, the plunger elements of the plunger arrangement are guided by a guide structure of the housing. For example, the guide structure may comprise a groove within the housing. For example, the plunger elements are at least partly arranged within the groove such that they may be rotated around the axis while being guided by the groove.

In one embodiment, the drug container is a syringe. In other embodiments, the drug container may be a cartridge. The drug container may comprise the needle, e.g. the first or second needle, or may be coupled with the needle, e.g. the first or second needle, manually before using the drug delivery device or automatically when using the drug delivery device.

We note that features described above and below in conjunction with different embodiments or aspects can be combined with one another, even if such a combination is not explicitly disclosed herein above or below. Further features, advantages and expediencies of the disclosure and, particularly, of the proposed concepts will become apparent from the following description of the exemplary embodiments in conjunction with the drawings.

### BRIEF DESCRIPTION OF THE DRAWINGS

FIG. 1 illustrates a top view of an interior of an exemplary embodiment of a drug delivery device.

FIG. 2 illustrates a side view of the interior of the drug delivery device of FIG. 1.

FIG. 3 illustrates an exemplary embodiment of a rotatable member of a drug delivery device.

FIG. 4 illustrates an exemplary embodiment of an energy storage member of a drug delivery device.

FIG. 5 illustrates another exemplary embodiment of a rotatable member of a drug delivery device.

FIG. 6 illustrates another exemplary embodiment of a rotatable member of a drug delivery device.

FIG. 7 illustrates the drug delivery device of FIG. 1 in a first state.

FIG. 8 illustrates the drug delivery device of FIG. 1 in a second state.

FIG. 9 illustrates the drug delivery device of FIG. 1 in a third state.

FIG. 10 illustrates the drug delivery device of FIG. 1 in a fourth state.

FIG. 11 illustrates a side view of another exemplary embodiment of a drug delivery device.

FIG. 12 illustrates an exemplary embodiment of a needle sleeve and a guide unit in a first state.

FIG. 13 illustrates the needle sleeve and the guide unit of FIG. 12 in a second state.

FIG. 14 illustrates the needle sleeve and the guide unit of FIG. 12 in a third state.

FIG. 15 illustrates the needle sleeve and the guide unit of FIG. 12 in a fourth state.

FIG. 16 illustrates the needle sleeve and the guide unit of FIG. 12 in a fifth state.

FIG. 17 illustrates the needle sleeve and the guide unit of FIG. 12 in a sixth state.

FIG. 18 illustrates an exemplary embodiment of a drug container.

FIG. 19 illustrates an exemplary embodiment of a drug container.

FIG. 20 illustrates an exemplary embodiment of a drug container.

FIG. 21 illustrates an expanded structural formula, molecular formula, and molecular weight of fitusiran.

### DETAILED DESCRIPTION

Identical elements, elements of the same kind and identically or similarly acting elements may be provided with the same reference numerals in the drawings.

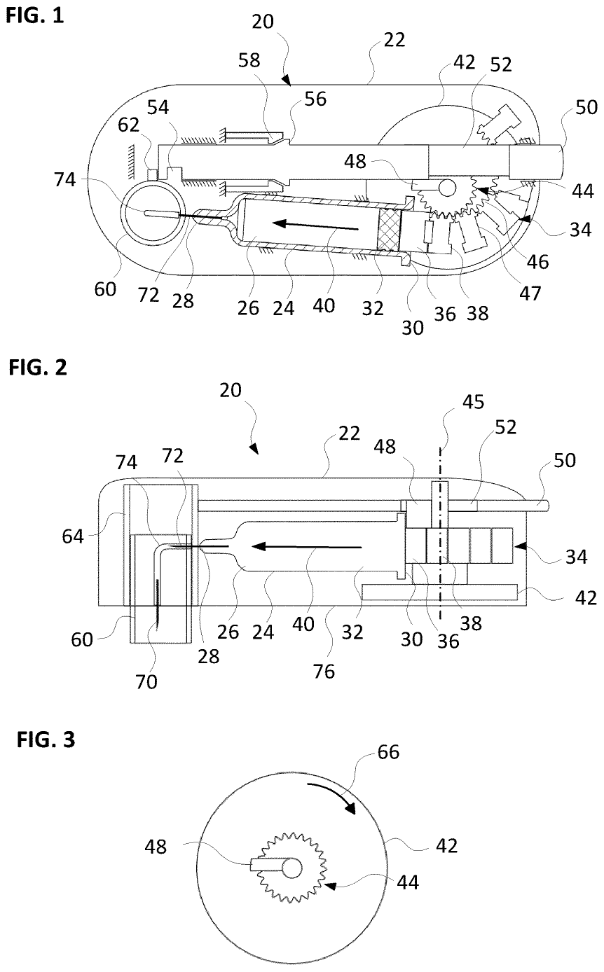

FIGS. 1 and 2 illustrate an embodiment of a drug delivery device 20. FIG. 1 illustrates a top view of an interior of an exemplary embodiment of a drug delivery device 20. FIG. 2 illustrates a side view of the interior of the drug delivery device 20 of FIG. 1. In particular, FIGS. 1 and 2 show the drug delivery device 100 in an initial or as delivered state.

The drug delivery device 20 comprises a housing 22. The housing 22 is provided to retain and/or retains a drug container 24 and a release mechanism of the drug delivery device 20 in its interior. A shape and/or size of the housing 22 may correspond to the shape and, respectively, size of a conventional (computer-)mouse.

The drug container 24 comprises a dispensing end 28 and a remote end 30 opposite the dispensing end 28. The drug container 24 comprises a chamber 26. A drug, i.e. a medicament, e.g. liquid medicament, may be arranged within the chamber 26. The chamber 26 is fluid-tight closed by a stopper 32. The stopper 32 is movably retained in the drug container 24 and may seal the drug container 24 remotely. The stopper 32 may be displaced towards an outlet of the drug container 24 at the dispensing end 28 to dispense the drug retained within the chamber 26 through the outlet. In particular, the stopper 32 may be movable in a dispensing direction 40 towards the dispensing end 28. If the stopper 32 is moved in the dispensing direction 40, the drug is dispensed through the outlet at the dispensing end 28.

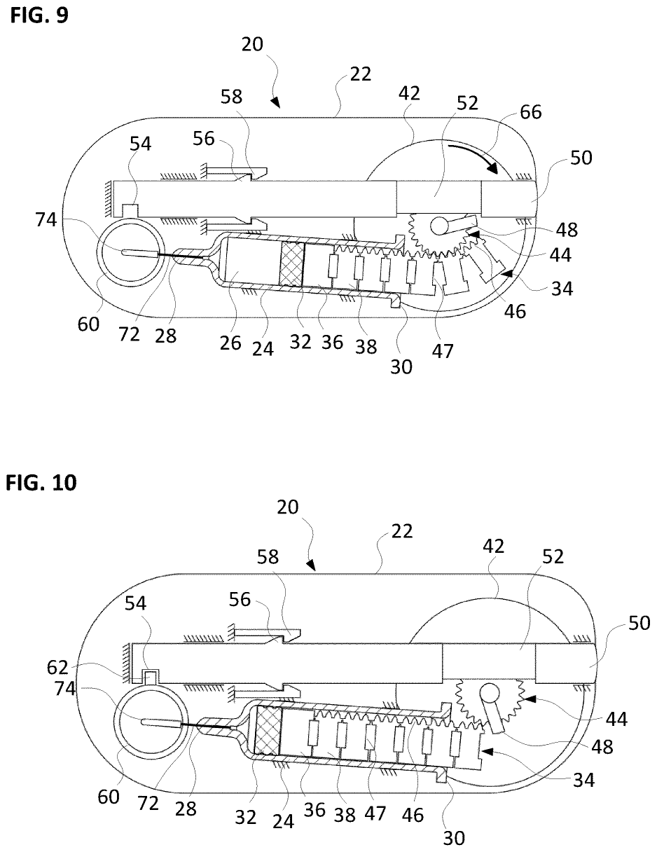

A drive mechanism provided to drive a drug delivery operation is expediently provided in the housing 22. The drive mechanism comprises a plunger arrangement 34, a rotatable member 42 and an energy storage member 86 (see FIG. 9). The plunger arrangement 34 comprises several plunger elements, in particular a first plunger element 36, a second plunger element 38, and further corresponding plunger elements, which are not characterized by corresponding reference numerals in the figures for clarity reasons. The first plunger element 36 may be in direct physical contact with the stopper 32 or, before the dispensing operation or delivery operation starts, there may be a gap between the first plunger element 36 and the stopper 32. The second plunger element 38 is in direct physical contact with the first plunger element 36. The first plunger element 36 is arranged between the stopper 32 and the second plunger element 38. The plunger elements 36, 38 may be coupled to each other such that they form a flexible plunger. For example, the plunger elements 36, 38 may be coupled to each other via hinges, in particular film hinges or living hinges. The plunger elements 36, 38 each may comprise a tooth portion 46 at a side of the corresponding plunger element 36, 38 facing inwardly.

The rotatable member 42 may comprise a gear wheel 44. The gear wheel 44 may be rigidly coupled to the rest of the rotatable member 42 such that a rotation of the rotatable member 42 corresponds to a rotation of the gear wheel 44. The gear wheel 44 may be configured to engage with the toothed portions 46 of the plunger elements 36, 38, at least upon rotation of the rotatable member 42. The toothed portions 46 may be formed at the sides of the plunger elements 36, 38 facing the axis 45. The plunger elements 36, 38 are coupled to the rotatable member 42 such that a rotation of the rotatable member 42 around an axis 45 may be transferred to a rotation of the plunger elements 36, 38 around the axis 45. If the rotatable member 42 is rotated each of the plunger elements 38 acts on the preceding plunger element 36, 38, wherein the first plunger element 36 acts on the stopper 32.

Alternatively or additionally to the gear wheel 44 and the toothed portions 46, the rotatable member 42 may comprise several protrusions 88 (see FIGS. 5 and 6) and the plunger elements 36, 38 each may comprise an indentation 47, wherein the protrusions 88 may be configured to engage with the indentations 47 such that the rotation of the rotatable member 42 is transferred to the plunger elements 36, 38 via the protrusions 88 and the corresponding indentations 47.

The energy storage member 86 is coupled to the rotatable member 42. In an initial state, the energy storage member 86 is loaded, wherein energy is stored in the loaded energy storage member 86. If the energy storage member 86 is released, the energy stored in the energy storage member 86 is released and transferred to the rotatable member 42 such that the rotatable member 42 is rotated. If the rotatable member 42 rotates, the rotation is transferred via the gear wheel 44 and the toothed portions 46 to the plunger elements 36, 38. Then, the plunger elements 36, 38 are introduced into the drug container 22 one after the other pushing the stopper 32 in the dispensing direction 40 and carrying out a drug delivery operation. In particular, the second plunger element 38 pushes the first plunger element 36 against the stopper 32 and the first plunger element 36 pushes the stopper 32 in the dispensing direction 40.

The rotatable member 42 may be coupled to the energy storage member 86 such that a locking of the rotatable member 42 corresponds to a locking of the energy storage member 86. So, if the rotatable member 42 is held in its initial state, the energy storage member 86 may be held in its initial state also. To hold the rotatable member 42 in its initial state, the rotatable member 42 may comprise an arrestor rib 48 which is blocked by a rod 50. The rod 50 may act as an activation member for activating the dispensing operation.

The rod 50 extends through the housing 22 in a direction perpendicular to the axis 45 and basically parallel to the dispensing direction 40. The rod 50 comprises the first recess 52, a second recess 54 and one or more first engaging features 56. The rod 50 remotely extends through the housing 22 such that the end of the rod 50 being exposed outside the housing 22 may be used as a button for pushing the rod 50 in the direction perpendicular to the axis 45 and basically parallel to the dispensing direction 40. The direction, in which the rod 50 may be moved, is not perfectly parallel to the dispensing direction 40. Therefore, in this description, the direction, in which the rod 50 may be moved, is referred to as “basically” the dispensing direction 40. The first recess 52 is arranged for accommodating the arrestor rib 48 of the rotatable member 42 in order to release the rotatable member 42 and as such the energy storage member 86, if the rod 50 is pushed basically in the dispensing direction 40. However, the movement of the rod 50 may be blocked by an arrestor pin 62 of a needle sleeve 60.

The needle sleeve 60 is arranged for protecting a needle of the drug delivery device 20, e.g. a first needle 70. The needle sleeve 60 protrudes from the housing 22 in the initial state. The needle sleeve 60 is movable relative to the housing 22 from an initial position or first position to a second position or trigger position. The needle sleeve 60 is movable along an axis that extends parallel to the axis 45. The needle sleeve 60 may be provided to extend beyond a tip of the first needle 70 which may protrude from the bottom of the housing 22 before the drug delivery operation is commenced. The needle sleeve 60 is movably arranged within a guide unit 64. In particular, the needle sleeve 60 may be moved parallel to the axis 45 and as such may be further introduced into the guide unit 64. During this movement, e.g. before the needle sleeve 60 reaches the second position, the first needle 70 may pierce the skin of the user. The guide unit 64 may be rigidly coupled to the housing 22 or may be a part of the housing 22. The needle sleeve 60 may be pushed into the guide unit 64 when the drug delivery device 20 is arranged on an injection site, e.g. the skin of the user. In particular, if the drug delivery device 20 is positioned on the skin with a bearing surface 76 at a bottom of the housing 22 touching the skin, the needle sleeve 60 is pushed into the housing 22. If the needle sleeve 60 is pushed into the housing 22, the first needle 70 is exposed and may pierce the skin. The needle 70 may not be movable relative to the housing 22 and/or the drug container 24. The needle 70 may not be able to move in a direction parallel to the axis 45. Moreover, the drug container 24 may not be movable relative to the housing 22.

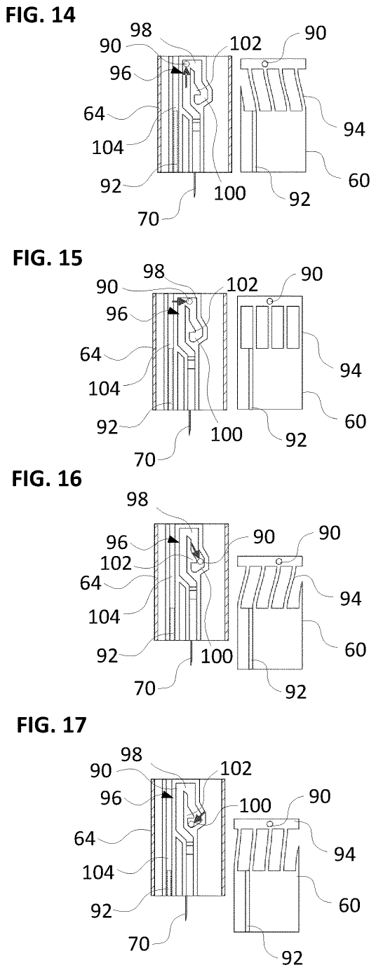

The needle sleeve 60 may serve as a trigger member of the drug delivery device 20. The needle sleeve 60 as trigger member, when displaced from the initial or first position depicted in FIGS. 1 and 2 to the second or trigger position (see FIGS. 3 and 14), may automatically initialize the drug delivery operation, preferably when it is in the second position. The needle sleeve 60, when moved from the first position to the second position and expediently when in the second position, may enable the triggering of the drug delivery operation via releasing the rod 50. In this case, the drug delivery operation may be initiated by pressing the trigger button, i.e. the remote end of the rod 50. Operating the trigger button to initiate the drug delivery operation may only be possible when the needle sleeve 60 is in the second position. In particular, if the needle sleeve 60 is pushed into the housing 22, the arrestor pin 62 may be moved outside of a plane in which the rod 50 is arranged and the rod 50 is not blocked by the arrestor pin 62 anymore. Then, the rod 50 may be pushed basically in the dispensing direction 40. So, the rod 50 may be moved only, if the drug delivery device 20 is positioned on the skin and the needle sleeve 60 exposes the first needle 70. If the rod 50 is pushed in its final position, in which the dispensing operation is completed, the first engaging feature of 56 may interact with one or more second engaging features 58 coupled to the housing 22 such that the rod 50 is held in its final position by the engaging features 56, 58.

In this embodiment, the needle sleeve 60 serves as the trigger member releasing the activation member, i.e. the rod 50, and the activation member has to be activated, e.g. pressed, to release the energy storage member 86 and thereby to start the dispensing operation. In other embodiments, the needle sleeve 60 may be configured as activation member. In this case, the drug delivery or dispensing operation may be triggered without pushing any further activation member, e.g. the rod 50, in addition to the needle sleeve 60 being moved into the housing 22. If the needle sleeve 60 serves as the activation member, the needle sleeve 60 may be mechanically and operatively coupled to the rotatable member 42 and/or the energy storage member 60 in order to retain the rotatable member 42 and/or the energy storage member 60 at first and then to release the rotatable member 42 and/or the energy storage member 60.

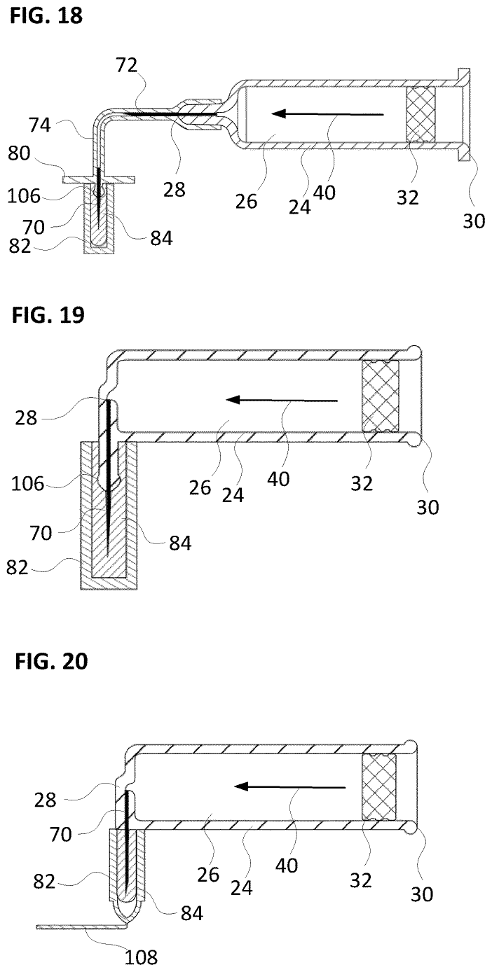

The first needle 70 may communicate with the drug container 24 via a conduit 74 and a second needle 72 of the drug container 24. The outlet of the drug container 24 at the dispensing end 28 may be formed or defined by the second needle 72. The second needle 72 may be an integral part of the drug container 24, e.g. (permanently or releasably) connected to a drug container body or separate from the drug container 24. In the first case, the drug container 24 may be a syringe. In the second case, the drug container 24 may be a cartridge. In case a cartridge is used as drug container 24, initially, the drug container 24 and the second needle 72 may be fluidly disconnected, and a fluid communication between an interior of the drug container 24 and the second needle 72 may be only established during operation of the drug delivery device 20. Further, instead of the two separate needles 70, 72 only one single needle may be arranged (see FIGS. 18 to 20).

The drug delivery device 20 is an autoinjector. The energy for driving the drug delivery operation in an autoinjector may be provided by components integral to the drug delivery device 20 and does not have to be loaded into the drug delivery device 20 by the user during the operation as is the case in many spring driven pen-type variable dose injectors, where, usually, the energy is loaded into a spring by the user during a dose setting procedure. The drug delivery device 20 expediently is a single shot device, i.e. it is provided to dispense only one dose. The drug delivery device 20 may be a disposable drug delivery device 20, that is to say a drug delivery device 20 which is disposed of after its use. The drug container 24 and/or the second needle 72 may be secured within the drug delivery device 20, e.g. within the housing 22. So, the user may have to perform the movement for piercing the skin with the first needle 70 communicating with the second needle 72 by placing the drug delivery device 20 on the skin.

FIG. 3 illustrates an exemplary embodiment of a rotatable member of a drug delivery device, e.g. the rotatable member 42 of the above drug delivery device 20. The rotatable member 42 comprises the gear wheel 44 and the arrestor rib 48. The rotatable member 42, the gear wheel 44 and the arrestor rib 48 are fixed to each other and rotatable around the same axis 45. A direction of rotation 66 during the dispensing operation is illustrated by a curved arrow.

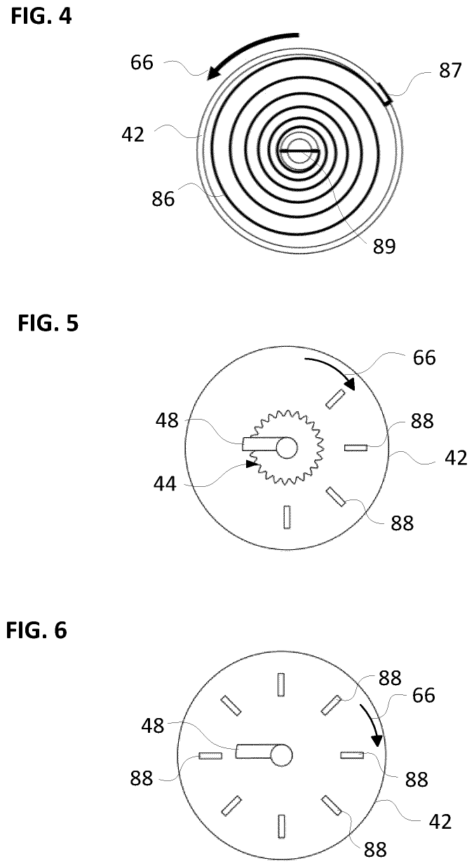

FIG. 4 illustrates an exemplary embodiment of an energy storage member of a drug delivery device, e.g. the energy storage member 86 of the above drug delivery device 20. The energy storage member 86 may be arranged at a bottom of the rotatable member 42 or in a recess of the rotatable member 42. So, FIG. 4 shows a bottom view of the rotatable member 42 shown in FIG. 3. In this embodiment, the energy storage member 86 is a spiral spring, in particular a flat spiral spring. The flat spiral spring comprises a hook 87 at one end of the flat spiral spring. The hook 87 may be hooked into a corresponding hook recess (not shown) of the rotatable member 42 such that the end of the flat spiral spring comprising the hook 87 is fixedly connected to the rotatable member 42. The other end of the flat spiral spring is connected to the axis 45 by a fixation 89. The axis 45 is fixed in this context. The fixation 89 may comprise a soldering or clamping of the flat spiral spring to the fixed axis 45.

In the initial state of the drug delivery device 20, the energy storage member 86 is loaded and as such the flat spiral spring is biased. If the rotatable member 42 is released, the flat spiral spring rotates the rotatable member 42 in the direction of rotation 66 and the rotatable member 42 rotates the plunger arrangement 34 and pushes the plunger elements 36, 38 in the dispensing direction against the stopper 32.

Other potential drive energy sources different from a spring comprise an electrical power cell or battery for driving the plunger arrangement 34 by a motor or a reservoir suitable to provide gas pressure, where the gas pressure can be used to drive the drug delivery operation.

FIG. 5 illustrates another exemplary embodiment of a rotatable member of a drug delivery device, e.g. the above rotatable member 42. The rotatable member 42 comprises several protrusions 88 at the surface of the rotatable member 42 facing the plunger arrangement 34. The protrusions 88 are formed and arranged such that they act on the indentations 47 of the plunger elements 36, 38, if the rotatable member 42 is rotated. In particular, the protrusions 88 engage with the indentations 47 at least in part and push the corresponding plunger elements 36, 38 in the direction of rotation 66.

FIG. 6 illustrates another exemplary embodiment of a rotatable member of a drug delivery device, e.g. the above rotatable member 42. The rotatable member 42 comprises the protrusions 88 at the surface of the rotatable member 42 facing the plunger arrangement 34. The protrusions 88 are formed and arranged such that they act on the indentations 47 of the plunger elements 36, 38, if the rotatable member 42 is rotated. In particular, the protrusions 88 engage with the indentations 47 at least in part and push the corresponding plunger elements 36, 38 in the direction of rotation 66. However, the rotatable member 42 shown in FIG. 6 does not comprise the gear wheel 44. Correspondingly, the plunger elements 36, 38 do not comprise the tooth portions 46 in this embodiment.

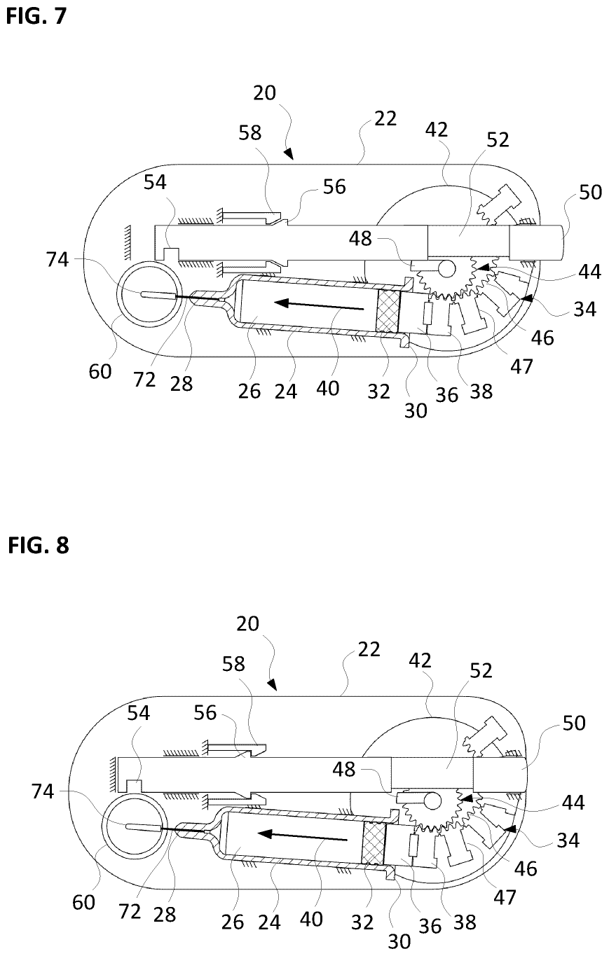

FIG. 7 illustrates the drug delivery device 20 of FIG. 1 in a first state. In the first state, the drug delivery device 20 is arranged on the skin such that the needle sleeve 60 is pushed into the housing 22, that the first needle 70 is exposed and that the arrestor pin 62 (see FIG. 1) is moved out of the plane of the rod 50. In the first state, the rod 50 may be pushed basically in the dispensing direction 40 by the user.

FIG. 8 illustrates the drug delivery device 20 of FIG. 1 in a second state. In the second state, the rod 50 is pushed basically in the dispensing direction 40 by the user such that the rod 50 is in its final position. In the final position of the rod 50, the first engaging features 56 engage with the second engaging features 58 and the rod 50 is held in its final position by the engaging features 56, 58. In addition, the arrestor rib 48 of the rotatable member 42 overlaps the first recess 52 of the rod 50 and as such is released. Then, the rotatable member 42 may be rotated by the energy storage member 86.

FIG. 9 illustrates the drug delivery device 20 of FIG. 1 in a third state. In the third state, the rotatable member 42 is rotated. The rotatable member 42 rotates the plunger elements 36, 38 of the plunger arrangement 34 around the axis 45 and pushes more and more of the plunger elements 36, 38 into the drug container 24 and against the stopper 32. The stopper 32 is pushed in the dispensing direction 40 by the plunger elements 36, 38 and presses the drug through the outlet of the drug container 24, in particular through the second needle 72, the conduit 74 and the first needle 70. So, in the third state, the dispensing operation is carried out and the drug is dispensed.

FIG. 10 illustrates the drug delivery device 20 of FIG. 1 in a fourth state. In the fourth state, nearly all plunger elements 36, 38 are introduced into the drug container 24 and the stopper 32 bumps against an inner end wall of the drug container 24. Therefore, the stopper 32 may not be moved further and no further drug may be dispensed through the needles 70, 72. So, in the fourth state, the dispensing operation is accomplished. Further, in the fourth state, the drug delivery device 20 may be removed from the skin of the user such that the needle sleeve 60 moves out of the housing 22, in particular out of the guide unit 64, and protects the first needle 70, and that the arrestor pin 62 moves into the second recess 54 of the rod 50 within the plane of the rod 50.

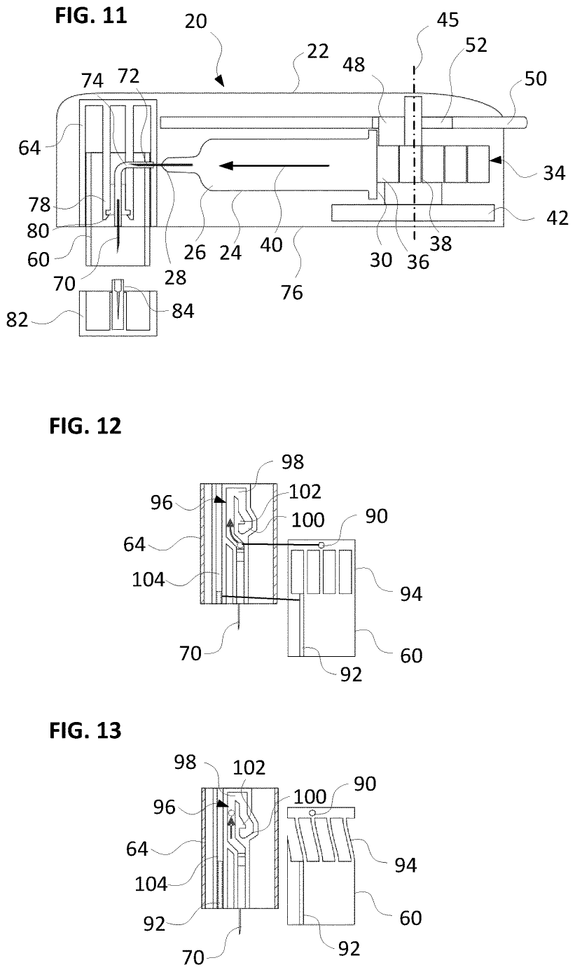

FIG. 11 illustrates a side view of another exemplary embodiment of a drug delivery device 20. The drug delivery device 20 may widely correspond to the above drug delivery device 20. Therefore, only those features of the drug delivery device 20 are explained in the following, in which the drug delivery device 20 shown in FIG. 11 differs from the above drug delivery device 20. The guide unit 64 of the drug delivery device 20 shown in FIG. 11 comprises arms 78 for holding the conduit 74. In this context, the conduit 74 may comprise a holder 80 which may be held by the arms 78. For example the arms 78 and the holder 80 are engaged with each other.

The drug delivery device 20 may comprise a cap 82. The cap 82 may be arranged at a dispensing end of the first needle 70. The cap 82 is detachably connected to the remainder of the drug delivery device 20, e.g. to the housing 22, the needle sleeve 60, the guide unit 64 and/or another component or member of the drug delivery device 20. The cap 82 may cover an opening of the needle sleeve 60 and/or a tip of the first needle 70. The cap 82 may comprise a needle shield 84, which covers the first needle 70 such that the needle shield 84 may be removed from the first needle 70 together with the cap 82, e.g. when the cap 82 is detached or disconnected from the drug delivery device 20.

FIG. 12 illustrates an exemplary embodiment of a needle sleeve and a guide unit in a first state, e.g. the above needle sleeve 60 and the above guide unit 64. In particular, FIG. 12 illustrates a side view of the needle sleeve 60 and a cutaway side view of the guide unit 64 in the first state. In the first state of the needle sleeve 60, the drug delivery device 20 is not yet arranged on the skin of the user and the needle sleeve 60 protects the first needle 70.

The needle sleeve 60 comprises a guide pin 90, a torsion protection 92, and flexible bars 94. The guide pin 90 protrudes from an outer wall of the needle sleeve 60 outwardly. The flexible bars 94 are separated by through-recesses extending through the wall of the needle sleeve 60. So, the flexible bars 94 and the body of the needle sleeve 60 may be made of one piece. The torsion protection 92 may comprise a bar extending vertically in FIG. 12.

The guide unit 64 comprises a first channel 96 and a second channel 104. The first channel 96 extends firstly with a slide inclination against the vertical direction and then basically verticality towards a bent 98 of the first channel 96 and then back towards a dead end 100 of the first channel 96. The dead end 100 is separated from the rest of the first channel 96 by a barb 102. In the first state of the needle sleeve 60, the guide pin 90 is arranged in a part of the first channel 96 below the dead end 100 and at the beginning of the inclination of the first channel 96.

The torsion protection 92 of the needle sleeve 60 is arranged within the second channel 104 and is guided by the second channel 104 during the movement of the needle sleeve 60 relative to the guide unit 64. The second channel 104 is straight and parallel to the axis 45 and as such parallel to the moving direction of the needle sleeve 60. The torsion protection 92 within the second channel 104 serves as a protection of the needle sleeve 60 against a rotation of the needle sleeve 60.

FIG. 13 illustrates the needle sleeve 60 and the guide unit 64 of FIG. 12 in a second state. In the second state of the needle sleeve 60, the needle sleeve 60 may be partly arranged within the guide unit 64, e.g. because of the drug delivery device 20 being partly arranged on the skin of the user. In the second state of the needle sleeve 60, the guide pin 90 is moved within the first channel 96 towards the bent 98. When the guide pin 90 passes the inclination of the first channel 96, an upper portion of the needle sleeve 60 is moved perpendicular to the moving direction of the needle sleeve 60 and the flexible bars 94 are flexed, because the rest of the needle sleeve 60 is secured against any rotation by the torsion protection 92 within the second channel 104. Then, the flexible bars 94 are biased.

FIG. 14 illustrates the needle sleeve 60 and the guide unit 64 of FIG. 12 in a third state. In the third state of the needle sleeve 60, the needle sleeve 60 is pressed into the guide unit 64 completely, e.g. because of the user arranging the drug delivery device 20 on his/her skin. So, in the third state of the needle sleeve 60, the first needle 70 is exposed by the needle sleeve 60. In this situation, the guide pin 90 has arrived in the bent 98 of the first channel 96 and may be moved perpendicular to the moving direction of the needle sleeve 60 within the bent 98. In the third state of the needle sleeve 60, the biased flexible bars 94 force the guide pin 90 through the bent 98.

FIG. 15 illustrates the needle sleeve 60 and the guide unit 64 of FIG. 12 in a fourth state. In the fourth state of the needle sleeve 60, the flexible bars 94 are released and the guide pin 90 has moved perpendicular to the moving direction of the needle sleeve 60 within the bent 98.

FIG. 16 illustrates the needle sleeve 60 and the guide unit 64 of FIG. 12 in a fifth state. In the fifth state of the needle sleeve 60, the drug delivery device 20 may be partly removed from the skin of the user. The guide pin 90 is forced over the barb 102 of the first channel 100 such that the flexible bars 94 are biased again. When the drug delivery device 20 is removed from the skin of the user, the needle sleeve 60 may be pushed out of the housing 22, for example by a conventional needle sleeve spring (not shown) such that the guide pin 90 is forced over the barb.

FIG. 17 illustrates the needle sleeve 60 and the guide unit 64 of FIG. 12 in a sixth state. In the sixth state of the needle sleeve 60, the drug delivery device 20 may be completely removed from the skin of the user. The needle sleeve 60 may completely cover the first needle 70. The guide pin 90 snaps into the dead end 100 of the first channel 96 such that the needle sleeve 60 is fixedly engaged to the guide unit 64.

FIG. 18 illustrates an exemplary embodiment of a drug container. The drug container 24 of FIG. 18 may widely correspond to the above drug container 24. Therefore, only those features of the drug container 24 are explained in the following, in which the drug container 24 shown in FIG. 18 differs from the above drug container 24.

The first needle 70 is protected by the cap 82 and a needle shield 84 within the cap 82. The conduit 74 may comprise a ball structure 106 and the needle shield 84 may comprise a corresponding ball recess, in which the ball structure 106 is accommodated. The ball structure 106 is form-fitted within the ball recess. This form-fit provides a predetermined resistance against the needle shield 84 and the cap 82 being removed from the first needle 70. The cap 82 and the needle shield 84 may be configured such that the needle shield 84 is removed from the first needle 70, when the cap 82 is removed.

FIG. 19 illustrates an exemplary embodiment of a drug container. The drug container 24 of FIG. 19 may widely correspond to the above drug container 24. Therefore, only those features of the drug container 24 are explained in the following, in which the drug container 24 shown in FIG. 19 differs from the above drug container 24. The drug container 24 shown in FIG. 19 comprises one needle, e.g. the first needle 70, only. The needle 70 extends parallel to the axis 45.

FIG. 20 illustrates an exemplary embodiment of a drug container. The drug container 24 of FIG. 20 may widely correspond to the drug container 24 of FIG. 19. Therefore, only those features of the drug container 24 are explained in the following, in which the drug container 24 shown in FIG. 20 differs from the drug container 24 of FIG. 19. The drug container 24 shown in FIG. 20 comprises a gripping structure 108 at the cap 82. The gripping structure 108 enables to comfortably and easily grip and remove the cap 82. The gripping structure 108 may be hingedly coupled to the cap 82.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of pharmaceutical formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug reservoir” adapted for use with a drug delivery device. The drug reservoir 101a may be, e.g., a cartridge, syringe, reservoir, or other solid or flexible vessel (bag) configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug reservoir may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)₂ fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Further examples of APIs for the prophylaxis of hemophilia A or B, with or without inhibitors, include an siRNA targeting antithrombin. An example of an siRNA targeting antithrombin is fitusiran. The term “prophylaxis” and “prophylactic treatment” are used interchangeably herein.

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, pharmaceutical formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### Fitusiran as the API for the Medicament in the Device

Fitusiran is a synthetic, chemically modified double-stranded small interfering RNA (siRNA) oligonucleotide covalently linked to a tri-antennary N-acetyl-galactosamine (GalNAc) ligand targeting AT3 mRNA in the liver, thereby suppressing the synthesis of antithrombin. See, e.g., Pasi et al., N Engl J Med. (2017) 377 (9): 819-28. The nucleosides in each strand of fitusiran are connected through either 3′-5′ phosphodiester or phosphorothioate linkages, thus forming the sugar-phosphate backbone of the oligonucleotide.

The sense strand and the antisense strand contain 21 and 23 nucleotides, respectively. The 3′ end of the sense strand is conjugated to the GalNAc containing moiety (referred to herein as L96) through a phosphodiester linkage. The sense strand contains two consecutive phosphorothioate linkages at its 5′ end. The antisense strand contains four phosphorothioate linkages, two at the 3′ end and two at the 5′ end. The 21 nucleotides of the sense strand hybridize with the complementary 21 nucleotides of the antisense strand, thus forming 21 nucleotide base pairs and a two-base overhang at the 3′-end of the antisense strand. See also U.S. Pat. Nos. 9,127,274, 11,091,759, US2020/0163987A1, and WO 2019/014187, the entire contents each of which are expressly incorporated herein by reference.

sense strand:
(SEQ ID NO: 1)
5′Gf-ps-Gm-ps-Uf-Um-Af-Am-Cf-Am-Cf-Cf-Af-Um-
Uf-Um-Af-Cm-Uf-Um-Cf-Am-Af-L96 3′,
and
antisense strand:
(SEQ ID NO: 2)
5′ Um-ps-Uf-ps-Gm-Af-Am-Gf-Um-Af-Am-Af-Um-Gm-
Gm-Uf-Gm-Uf-Um-Af-Am-Cf-Cm-ps-Am-ps-Gm 3′,

wherein
Af=2′-deoxy-2′-fluoroadenosine
Cf=2′-deoxy-2′-fluorocytidine
Gf=2′-deoxy-2′-fluoroguanosine
Uf=2′-deoxy-2′-fluorouridine
Am=2′-O-methyladenosine
Cm=2′-O-methylcytidine
Gm=2′-O-methylguanosine
Um=2′-O-methyluridine
“-” (hyphen)=3′-5′ phosphodiester linkage sodium salt
“-ps-”=3′-5′ phosphorothioate linkage sodium salt
   and wherein L96 has the following formula:

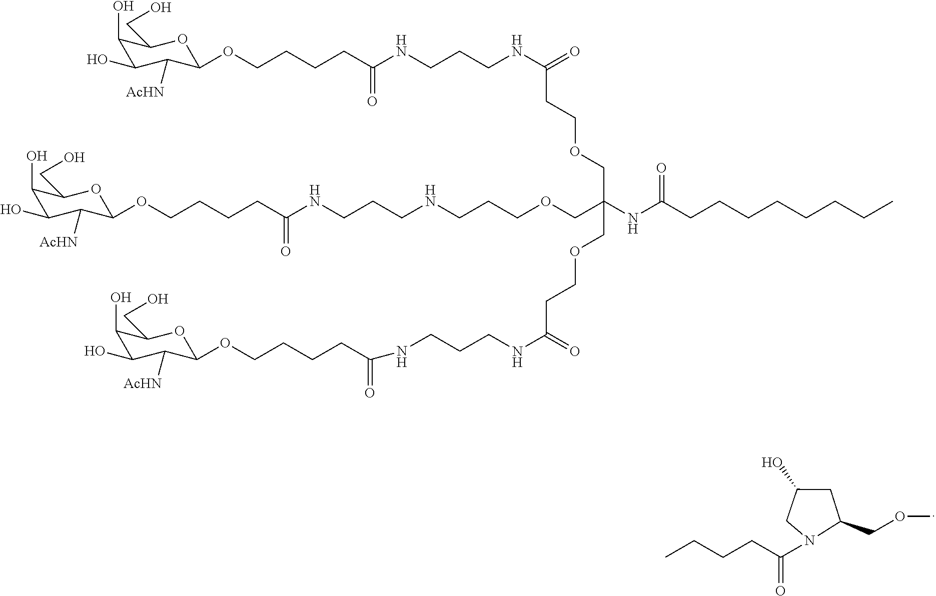

As used herein, the terms 2′-deoxy-2′-fluoroadenosine and 2′-fluoroadenosine may be used interchangeably.

As used herein, the terms 2′-deoxy-2′-fluorocytidine and 2′-fluorocytidine may be used interchangeably. As used herein, the terms 2′-deoxy-2′-fluoroguanosine and 2′-fluoroguanosine may be used interchangeably. As used herein, the terms 2′-deoxy-2′-fluorouridine and 2′-fluorouridine may be used interchangeably.

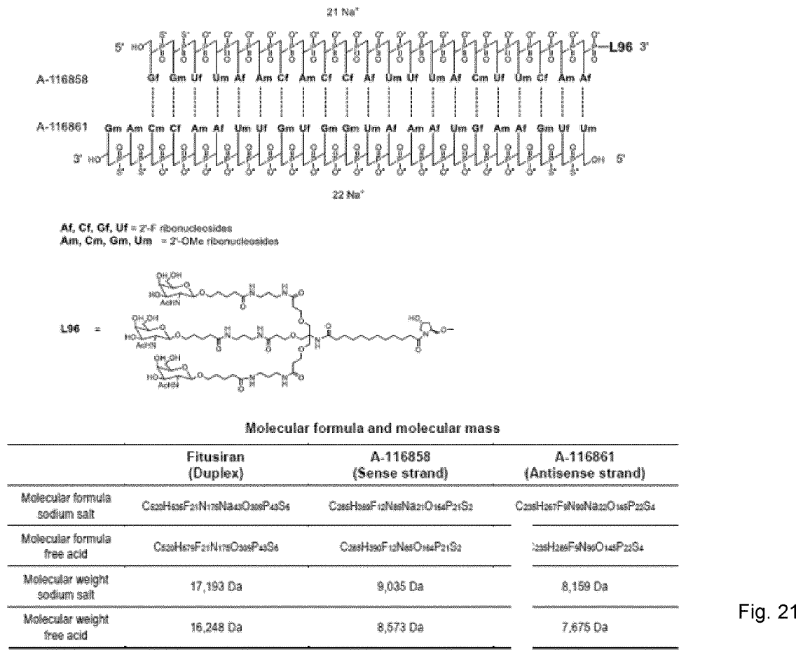

The expanded structural formula, molecular formula, and molecular weight of fitusiran are shown in FIG. 21.

The structure of fitusiran can also be described using the following diagram, wherein the X is O:

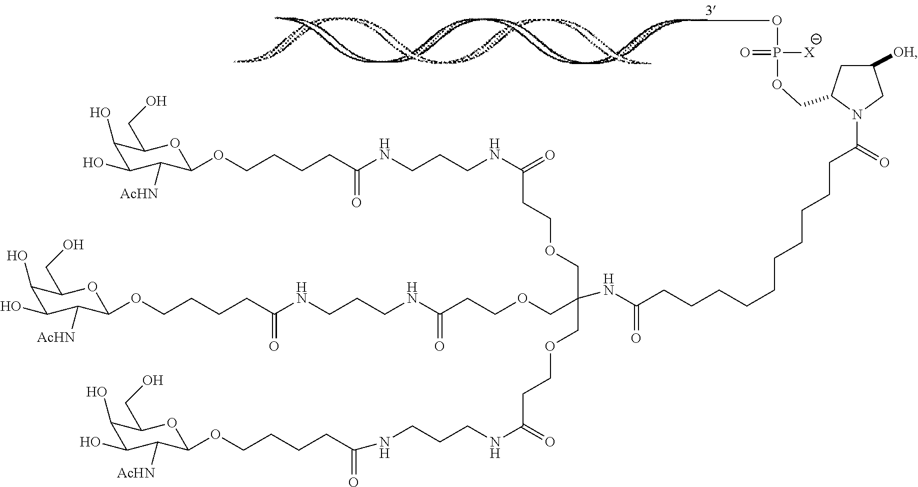

Fitusiran is shown in FIG. 21 in sodium salt form.

In some embodiments, the device delivers fitusiran in an aqueous solution, wherein fitusiran is at a concentration of about 40 to about 200 mg/ml (e.g., about 50 to about 150 mg/mL, about 80 to about 110 mg/mL, or about 90 to about 110 mg/mL). As used herein, values intermediate to recited ranges and values are also intended to be part of this disclosure. In addition, ranges of values using a combination of any of recited values as upper and/or lower limits are intended to be included. In further embodiments, the pharmaceutical formulation comprises fitusiran in an aqueous solution at a concentration of about 40, about 50, about 75, about 100, about 125, about 150, or about 200 mg/mL. In certain embodiments, fitusiran is provided in an aqueous solution at a concentration of about 100 mg/mL.

The term “deliver,” “delivers,” or “delivering” is intended to mean “administer,” “administers,” or “administering.”

Unless specifically stated or otherwise evident from the context, as used herein, the term “approximately” or “about” refers to a value that is within an acceptable error range for a particular value determined by a person of ordinary skill, a portion of which will depend on how the measurement or determination is made. For example, “approximately” or “about” may mean a range of up to 10% (ie, ±10%). Therefore, “approximately” or “about” can be understood as greater than or less than 10%, 9%, 8%, 7%, 6%, 5%, 4%, 3%, 2%, 1%, 0.5%, 0.1%, 0.05%, 0.01%, or 0.001%. When a specific value is provided in this disclosure, unless otherwise stated, the meaning of “approximately” or “about” should be assumed to be within an acceptable error range for that specific value.

While the fitusiran dosage weight described herein refers to the weight of fitusiran free acid (active moiety), administration of fitusiran to patients herein refers to administration of fitusiran sodium (drug substance) provided in a pharmaceutically suitable aqueous solution (e.g., a phosphate-buffered saline at a physiological pH). For example, about 100 mg/mL fitusiran means about 100 mg of fitusiran free acid (equivalent to about 106 mg fitusiran sodium, the drug substance) per mL. Unless otherwise indicated, a fitusiran weight recited in the present disclosure is the weight of fitusiran free acid (the active moiety).

In some embodiments, a pharmaceutical formulation in the device comprises fitusiran in a phosphate-buffered saline. The phosphate concentration in the solution may be about 1 to about 10 mM (e.g., about 2, about 3, about 4, about 5, about 6, about 7, about 8, or about 9 mM), with a pH of about 6.0-8.0. The pharmaceutical formulations herein may include a stabilizing agent such as EDTA. The pharmaceutical formulations may be preservative-free. In some embodiments, the fitusiran pharmaceutical formulation in the device is preservative-free and comprises, consists of, or consists essentially of about 100 mg of fitusiran per mL of an approximately 5 mM phosphate buffered saline (PBS) solution. In some embodiments, the fitusiran pharmaceutical formulation in the device is preservative-free and comprises, consists of, or consists essentially of fitusiran in an approximately 5 mM phosphate buffered saline (PBS) solution. The PBS solution is composed of sodium chloride, dibasic sodium phosphate (heptahydrate), and monobasic sodium phosphate (monohydrate). Sodium hydroxide solution and diluted phosphoric acid may be used to adjust the pH of the pharmaceutical formulation to about 7.0 or about 7.1.

In some embodiments, the fitusiran pharmaceutical formulation in the device for subcutaneous delivery contains fitusiran in a 5 mM phosphate buffered saline having 0.64 mM NaH₂PO₄, 4.36 mM Na₂HPO₄, and 84 mM NaCl at pH 7.0. In certain embodiments, the pharmaceutical formulation of fitusiran solution for subcutaneous delivery is shown in Table 1 below:

TABLE 1
Exemplary Fitusiran Pharmaceutical Formulation

Pharmaceutical Formulation

| | Percentage | Per ml |
|---|---|---|
| Components | [%] | [mg] |
| Fitusiran (active moiety) | 10 | 100 |
| [equivalent to fitusiran sodium] | | [106] |
| Sodium chloride | 0.49 | 4.909 |
| Dibasic sodium phosphate | 0.12 | 1.169 |
| (heptahydrate) | | |
| Monobasic sodium phosphate | <0.01 | 0.0885 |
| (monohydrate) | | |
| Phosphoric acid, concentrated | — | q.s. pH 7.0 |
| Sodium hydroxide | — | q.s. pH 7.0 |
| Water for subcutaneous | q.s. 100 | q.s. 1 mL |
| delivery | | |

*q.s.: quantum satis

In some embodiments, the pharmaceutical formulation of fitusiran solution for subcutaneous delivery with the device can be described as shown in Table 2 below.

TABLE 2
Exemplary Fitusiran Pharmaceutical Formulation

| | Pharmaceutical Formulation |
|---|---|
| Components | (mg) |
| Fitusiran (active moiety) | 100 |
| [equivalent to fitusiran sodium] | [106] |
| NaH₂PO₄*H₂O | 0.0885 |
| Na₂HPO₄*7H₂O | 1.169 |
| NaCl | 4.909 |
| 0.1N NaOH | q.s. |
| 0.1M H₃PO₄ | q.s. |
| Purified water | Ad 1 mL |

In some embodiments, the device may be used to deliver a single dose of fitusiran wherein the single dose comprises about 20 to about 80 mg of fitusiran (e.g., about 20 mg, about 25 mg, about 30 mg, about 40 mg, about 50 mg, or about 80 mg). In some embodiments, the device may be used to deliver single dose of fitusiran, wherein the single dose comprises about 1 to about 30 mg of fitusiran (e.g., about 1.25 mg, about 2.5 mg, about 5 mg, about 10 mg, about 20 mg, or about 30 mg).

In one embodiment, the device may be used to deliver a single dose of about 80 mg of fitusiran. In one embodiment, the device may be used to deliver a single dose of about 50 mg of fitusiran. In one embodiment, the device may be used to deliver a single dose of about 20 mg of fitusiran. In one embodiment, the device may be used to deliver a single dose of about 30 mg of fitusiran. In one embodiment, the device may be used to deliver a single dose of about 10 mg of fitusiran. In one embodiment, the device may be used to deliver a single dose of about 5 mg of fitusiran. In one embodiment, the device may be used to deliver a single dose of about 2.5 mg of fitusiran. In one embodiment, the device may be used to deliver a single dose of about 1.25 mg of fitusiran.

In some embodiments, the single dose of fitusiran may be delivered in about 0.5 mL to about 1 mL delivery volumes (e.g., about 0.5 mL, about 0.6 mL, about 0.7 mL, about 0.8 mL, about 0.9 mL, or about 1 mL). Other delivery volumes described herein may also be used.

In one embodiment, the device may be used to deliver a single dose of about 80 mg of fitusiran in about 0.8 mL (about 100 mg fitusiran/mL). In one embodiment, the device may be used to deliver a single dose of about 50 mg of fitusiran in about 0.5 mL (about 100 mg fitusiran/mL). In one embodiment, the device may be used to deliver a single dose of about 20 mg of fitusiran in about 0.5 mL (about 40 mg fitusiran/mL). In one embodiment, the device may be used to deliver a single dose of about 30 mg of fitusiran in about 0.5 mL (about 60 mg fitusiran/mL). In one embodiment, the device may be used to deliver a single dose of about 10 mg of fitusiran in about 0.5 mL (about 20 mg fitusiran/mL). In one embodiment, the device may be used to deliver a single dose of about 5 mg of fitusiran in about 0.5 mL (about 10 mg fitusiran/mL). In one embodiment, the device may be used to deliver a single dose of about 2.5 mg of fitusiran in about 0.5 mL (about 5 mg fitusiran/mL). In one embodiment, the device may be used to deliver a single dose of about 1.25 mg of fitusiran in about 0.5 mL (about 2.5 mg fitusiran/mL).

In one embodiment, the device delivers fitusiran at a prophylactically effective amount to prophylactically treat hemophilia (e.g., hemophilia A or B, in a patient with or without inhibitors) in a patient in need thereof (e.g., a hemophilia A or B patient, with or without inhibitors). “Prophylactically effective amount” refers to the amount of fitusiran that helps the patient with hemophilia A or B, with or without inhibitors to achieve a desired clinical endpoint such as reducing the Annualized Bleeding Rate (ABR), Annualized Joint Bleeding Rate (AjBR), Annualized Spontaneous Bleeding Rate (AsBR), or the frequency of bleeding episodes. As used herein in the context of fitusiran, the term “treat” “treating,” or “treatment” includes prophylactic treatment of the disease and refers to achievement of a desired clinical endpoint.

A hemophilia A or B patient with inhibitors refers to a patient who has developed alloantibodies to the factor he/she has previously received (e.g., factor VIII for hemophilia A patients or factor IX for hemophilia B patients). A hemophilia A or B patient with inhibitors may become refractory to replacement coagulation factor therapies. A patient without inhibitors refers to a patient who does not have such alloantibodies. The present treatment methods may be beneficial for hemophilia A patients with inhibitors, as well as for hemophilia B patients with inhibitors.

As used herein, a patient with “hemophilia A or B, with or without inhibitors,” or refers to 1) a hemophilia A patient with inhibitors, or 2) a hemophilia B patient with inhibitors, 3) a hemophilia A patient without inhibitors, or 4) a hemophilia B patient without inhibitors. As used herein, a patient refers to a human patient. A patient can also refer to a human subject.

In some embodiments, the device may be used to prophylactically treat a patient with hemophilia A or B, with or without inhibitors, with a subcutaneous dose of about 50 mg of fitusiran once every two months (or every eight weeks). In other embodiments, the device may be used to prophylactically treat a patient with hemophilia A or B, with or without inhibitors, with a subcutaneous dose of about 50 mg of fitusiran every month (or every four weeks). In yet other embodiments, the device may be used to prophylactically treat a patient with hemophilia A or B, with or without inhibitors, with a subcutaneous dose of about 80 mg of fitusiran every two months (or every eight weeks). In yet other embodiments, the device may be used to prophylactically treat a patient with hemophilia A or B, with or without inhibitors, with a subcutaneous dose of about 80 mg of fitusiran every month (or every four weeks). In yet other embodiments, the device may be used to prophylactically treat a patient with hemophilia A or B, with or without inhibitors, with a subcutaneous dose of about 20 mg of fitusiran every two months (or every eight weeks). In yet other embodiments, the device may be used to prophylactically treat a patient with hemophilia A or B, with or without inhibitors, with a subcutaneous dose of about 20 mg of fitusiran every month (or every four weeks). In yet other embodiments, the device may be used to prophylactically treat a patient with hemophilia A or B, with or without inhibitors, with a subcutaneous dose of about 10 mg of fitusiran every month (or every four weeks). In yet other embodiments, the device may be used to prophylactically treat a patient with hemophilia A or B, with or without inhibitors, with a subcutaneous dose of fitusiran at about 30 mg every month (or every four weeks). In yet other embodiments, the device may be used to prophylactically treat a patient with hemophilia A or B, with or without inhibitors, with a subcutaneous dose of fitusiran at about 5 mg every month (or every four weeks). In yet other embodiments, the device may be used to prophylactically treat a patient with hemophilia A or B, with or without inhibitors, with a subcutaneous dose of fitusiran at about 2.5 mg every month (or every four weeks). In yet other embodiments, the device may be used to prophylactically treat a patient with hemophilia A or B, with or without inhibitors, with a subcutaneous dose of fitusiran at about 1.25 mg every month (or every four weeks).

Accordingly, provided herein is a method of prophylactic treatment of a patient with hemophilia A or hemophilia B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of prophylactic treatment of a patient with hemophilia A or hemophilia B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Further provided herein is a method of reducing the frequency of bleeding episodes in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the frequency of bleeding episodes in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Also, provided herein is a method of reducing the ABR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the ABR in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Also, provided herein is a method of reducing the AjBR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). The fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the AjBR in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Also, provided herein is a method of reducing the AsBR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the AsBR in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Any invention described herein is not limited by the description in conjunction with the exemplary embodiments. Rather, the invention and the associated disclosure comprise any new feature as well as any combination of features, particularly including any combination of features in the patent claims, even if said feature or said combination per se is not explicitly stated in the patent claims or exemplary embodiments.

### REFERENCE NUMERALS

20 drug delivery device
22 housing
24 drug container
26 chamber
28 dispensing end
30 remote end
32 stopper
34 plunger arrangement
36 first plunger element
38 second plunger element
40 dispensing direction
42 rotatable member
44 gear wheel
45 axis
46 toothed portion
47 indentation
48 arrestor rib
50 rod
52 first recess
54 second recess
56 first engaging feature
58 second engaging feature
60 needle sleeve
62 arrestor pin
64 guide unit
66 direction of rotation
70 first needle
72 second needle
74 conduit
76 bearing surface
78 arms
80 holder
82 cap
84 needle shield
86 energy storage member
87 hook
88 protrusion
89 fixation
90 guide pin
92 torsion protection
94 flexible bars
96 first channel
98 bend
100 dead end
102 barb
104 second channel
106 ball structure
108 gripping structure

1-18. (canceled)

19. A drug delivery device, comprising:
a housing;
a drug container within the housing, the drug container having a chamber, a dispensing end, and a remote end facing away from the dispensing end;
a stopper within the chamber, wherein the stopper sealingly closes the chamber of the drug container on the side of the remote end of the drug container; and
a plunger arrangement having several plunger elements for moving the stopper in a dispensing direction towards the dispensing end of the drug container,
wherein the plunger elements are configured for being rotated around an axis perpendicular to the dispensing direction, for being introduced into the chamber of the drug container one after the other and for pushing the stopper in the dispensing direction, upon rotation of the plunger elements around the axis.

20. The drug delivery device of claim 19, further comprising a drug within the chamber, wherein the drug is enclosed by the drug container and the stopper.

21. The drug delivery device of claim 20, further comprising a needle for injecting the drug into an injection site, and a needle sleeve for protecting the needle.

22. The drug delivery device of claim 21, wherein the needle sleeve is movable parallel to the axis.

23. The drug delivery device of claim 21, wherein the drug delivery device is configured such that when the needle sleeve is displaced from an initial position to a trigger position, the drug delivery device automatically initializes the drug delivery operation.

24. The drug delivery device of claim 21, wherein the needle is communicatively coupled to the drug container at the dispensing end of the drug container.

25. The drug delivery device of claim 24, wherein the needle extends in a direction parallel to the axis.

26. The drug delivery device of claim 19, wherein the plunger elements are configured such that a first plunger element of the plunger elements pushes the stopper in the dispensing direction and that a second plunger element of the plunger elements pushes the first plunger element towards the stopper, upon rotation of the plunger elements around the axis.

27. The drug delivery device of claim 19, wherein the plunger elements are coupled to each other at sides of the plunger elements facing the axis.

28. The drug delivery device of claim 19, further comprising a rotatable member, which is rotatable around the axis and which is coupled to the plunger elements such that the rotatable member rotates the plunger elements around the axis upon rotation of the rotatable member.

29. The drug delivery device of claim 28,
wherein the rotatable member comprises a gear wheel; and
wherein the plunger elements each comprise a toothed portion configured to engage with the gear wheel upon rotation of the rotatable member around the axis.

30. The drug delivery device of claim 28,
wherein the rotatable member comprises several protrusions at a surface of the rotatable member, with the surface facing the plunger elements and being perpendicular to the axis, and
wherein the plunger elements each comprise an indentation, with the protrusions of the rotatable member being configured to engage with the indentation of the corresponding plunger element, upon rotation of the rotatable member around the axis.

31. The drug delivery device of claim 28, further comprising an energy storage member coupled to the rotatable member and configured for rotating the rotatable member, wherein the energy storage member is loaded in an initial state.

32. The drug delivery device of claim 31, further comprising a release mechanism being configured for locking the rotatable member in the initial state and for releasing the rotatable member such that the energy storage member rotates the rotatable member around the axis, upon activation of the release mechanism.

33. The drug delivery device of claim 32, wherein the release mechanism comprises a rod, which is configured for blocking an arrestor rib of the rotatable member in the initial state and for releasing the arrestor rib upon moving the rod.

34. The drug delivery device of claim 32, wherein the needle sleeve is operatively coupled to the release mechanism and configured such that an activation of the release mechanism is prevented as long as the needle sleeve protects the needle and that the activation of the release mechanism is enabled, when the needle sleeve exposes the needle.

35. The drug delivery device of claim 32, further comprising one or more engaging features configured to engage with the release mechanism and to lock the release mechanism in a final state after the drug was injected.

36. The drug delivery device of claim 19, wherein the plunger elements of the plunger arrangement are guided by a guide structure of the housing.

37. The drug delivery device of claim 19, wherein the drug container is a syringe.

38. The drug delivery device of claim 21, wherein the needle sleeve is movably arranged within a guide unit.

39. A method for dispensing a drug, comprising:
providing a drug delivery device, comprising:
a housing;
a drug container within the housing, the drug container having a chamber, a dispensing end, and a remote end facing away from the dispensing end;
a stopper within the chamber, wherein the stopper sealingly closes the chamber of the drug container on the side of the remote end of the drug container;
a drug contained within the chamber, wherein the drug is enclosed by the drug container and the stopper; and
a plunger arrangement having several plunger elements for moving the stopper in a dispensing direction towards the dispensing end of the drug container,
wherein the plunger elements are configured for being rotated around an axis perpendicular to the dispensing direction, for being introduced into the chamber of the drug container one after the other and for pushing the stopper in the dispensing direction, upon rotation of the plunger elements around the axis, and
causing the plunger elements to rotate around the axis.
